(19) United States
(12) Patent Application Publication
(10) Pub. No.: US 2008/0194904 A1
(43) Pub. Date: Aug. 14, 2008

(54) TO TREAT HUMANS AND ALL LIVING ANIMALS OR CREATURES

(76) Inventor: HOWARD ORVILLE RHINE, Kirkland, WA (US)

Correspondence Address: MICHAEL RIES, 318 PARKER PLACE, OSWEGO, IL, 60543, US

(21) Appl. No.: 12/030,137

(22) Filed: Feb. 12, 2008

Related U.S. Application Data
(60) Provisional application No. 60/900,712, filed on Feb. 12, 2007.

(51) Int. Cl. A61N 2/00; A61B 5/00

(52) U.S. Cl. 600/9; 600/300

(57) ABSTRACT

The present invention is a method to treat humans or animals by holding a vial or food item or supplement in a glass or plastic container or by itself held in the left or right hand or both hands of a human or tape the vial or food to an animal. This method will treat both bad and good in the body; you can treat with anything and everything.

This application claims priority to U.S. Provisional Application 60/900,712 filed Feb. 12, 2007, the entire disclosure of which is incorporated by reference.

### TECHNICAL FIELD & BACKGROUND

The present invention generally relates to treating animals or living creatures. More specifically, the present invention is a method to treat a human or animal back to health.

The present invention is a method to treat by holding a vial or food item or supplement in a glass or plastic container or just holding item alone being held in the left or right hand or both hands of a human. This method will treat both bad and good in your body you can treat with anything and everything. You remove the bad first before you can get the good in there are exceptions.

### DETAILED DESCRIPTION

The present invention can be done by holding the item in your left hand this is better because your pulling everything through your body instead of pushing everything, through organs and body. The treatment should be tailored to individuals history. You can treat past or present items or continue to treat as the doctor orders. You can take supplements by the mouth while being treated; supplements are stronger while taking and treating this also applies to herbs. You can add stimulation to any part of your body by a magnet or ionized bracelet. Do not get a magnet to close to other stimulators, electrodes or neuro-stimulator or anything that will stimulate the body. Acupuncture will do the same thing. Any electrical device can destroy any magnetic imprint on vials. You can add more than one type of stimulator together using more then one and add anything else even though it does nothing more as far as stimulation with the other items.

You should treat addictions first together with other diseases. Sexual addiction should be treated with all problems and you should abstain from sex while being treated no matter what the other problem is. If a doctor is treating with antibiotic or something that has a similar quality and anything that will relax the body many different herbs. These items meet the exception.

Do not use stimulation until you have used other types of treatment because a stimulator renews your body to that object as if you never have touched it or held it before. In any substance food or vegetable item there are many different nutrients or all plants anything poisonous or not can be used so take particular supplement or nutrient minerals, vitamins or anything from a health store or available from doctors. Anything that has value you don't eat it only but are taking the substance as a supplement you are taking all that has value from the substance that you are holding or being treated with such as vitamins, protean that is inside the substance or item being held. Anything can be used. Most all of life has lecithin in it so I like taking it as a supplement with other supplements and nutrients.

Treat all till completed you will know when all the toxins come out of your body and lymph nodes of your body be careful when using stimulation skin may break out.

Before finishing with all treatments treat the whole body with other body parts including all body organs such as pancreas islets and take and treat with yogurt. Anything you take you can become immune to unless it is in your body. To treat animals it can be done the same way as humans but when placing a stimulator or vial item place by taping make sure you shave the area, both items will be taped. Place the item that is being treated and make sure you treat with animal organs, place item close to the animal or on leg and the stimulator close to the heart but not together. When the item is on the leg use stimulator elsewhere.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

What is claimed is:

1. A method comprising:
analyzing a patent's history of health problems;
treating the problems by determining causes of the problem;
placing an item in the patient's left hand the item has qualities that will resolve the patent's problem; and
balancing functions of the patent by the qualities of the item moving into the patent's body or out of the patent's body.

2. The method of claim 1 wherein taking a nutrient by mouth while holding the item, the nutrient will contain that same qualities to resolve the patient's problem.

3. The method of claim 1 wherein taking supplements by the mouth while being treated; supplements are stronger while taking and treating this also applies to herbs.

4. The method of claim 1 wherein adding stimulation to any part of your body by a selected one of magnet and ionized bracelet.
